(19) United States
(12) Patent Application Publication
(10) Pub. No.: US 2025/0050347 A1
(43) Pub. Date: Feb. 13, 2025

(54) COUPLING DEVICE

(71) Applicant: VETTER PHARMA-FERTIGUNG GMBH & CO. KG, Ravensburg (DE)

(72) Inventors: Michael WOLFF, Gestratz (DE); Lukas KROPP, Baienfurt (DE)

(21) Appl. No.: 18/723,090

(22) PCT Filed: Jan. 19, 2023
(86) PCT No.: PCT/EP2023/051282

§ 371 Date: Jun. 21, 2024

(30) Foreign Application Priority Data
Jan. 21, 2022 (DE) 10 2022 101 475.4

(51) Int. Cl. B01L 3/00

(52) U.S. Cl. CPC B01L 3/563; B01L 2400/0622

(57) ABSTRACT

The invention relates to a coupling device with
at least one first connector, at least one second connector, at least one third connector and a valve device, wherein
the at least one first connector is adapted to fluidically connect a media supply filter test device to the coupling device, wherein
the at least one second connector is adapted to fluidically connect a multiplier device to the coupling device, wherein
the at least one third connector is configured as a hot steam connector, wherein
the at least one first connector, the at least one second connector and the at least one third connector are flow connected to the valve device, wherein
the valve device is adapted to connect, in a first operating mode, at least one third connector of the at least one third connector fluidically simultaneously to at least one first connector of the at least one first connector and to at least one second connector of the at least one second connector, so that the media supply filter test device connected to the coupling device and the multiplier device connected to the coupling device can be sanitized simultaneously by means of the hot steam supplied through the at least one third connector.

### CROSS-REFERENCE TO RELATED APPLICATIONS

This application is a U.S. National Phase Application under 35 U.S.C. 371 of International Application No. PCT/EP2023/051282, filed on Jan. 19, 2023, which claims the benefit of German Patent Application No. 10 2022 101 475.4, filed on Jan. 21, 2022. The entire disclosure of the above patent application is incorporated herein by reference.

### FIELD

The invention relates to a coupling device.

### BACKGROUND

This section provides background information related to the present disclosure which is not necessarily prior art.

It is known that filters, in particular reusable filters, in particular reusable hydrophobic media filters, are subjected to a filter test by means of a media supply filter test device, in particular by means of an AquaWIT device from the company PALL, in order to verify the integrity of the filter. In the filter test, in particular a so-called water intrusion test (WIT), the filter is completely filled with water in order to create a water column in the filter. The water column is then subjected to a predetermined pressure and a water evaporation flow and/or water flow through a filter membrane is determined. If a predetermined water evaporation flow and/or water flow associated with a membrane is exceeded, the integrity of the filter cannot be guaranteed.

Furthermore, it is known that for a simultaneous connection of a plurality of filters for sequential testing of the plurality of filters, the media supply filter test device, in particular the AquaWIT device, is fluidically coupled to a multiplier device, in particular a MUX device from PALL. The multiplier device is adapted to accommodate a plurality of filters simultaneously.

Both the media supply filter test device, in particular the AquaWIT device, and the multiplier device, in particular the MUX device, should be sanitized at regular intervals in order to reduce the bacterial load in valves and/or media lines of the devices. In order to be able to carry out the sanitization, the fluidic connection between the media supply filter test device, in particular the AquaWIT device, and the multiplier device, in particular the MUX device, must be disconnected. The devices can then be connected individually, in particular to a sanitizing device, and sanitized. The disadvantage of this is that the devices can only be sanitized individually and that fluidic coupling and/or decoupling of the devices is very time-consuming and error-prone.

### SUMMARY

This section provides a general summary of the disclosure, and is not a comprehensive disclosure of its full scope or all of its features.

The invention is therefore based on the task of creating a coupling device, wherein the aforementioned disadvantages are at least partially eliminated, preferably avoided.

The task is solved by providing the present technical teaching, in particular the teaching of the independent claims and the embodiments disclosed in the dependent claims and the description.

In particular, the task is solved by providing a coupling device comprising at least one first connector, at least one second connector, at least one third connector and a valve device. The at least one first connector is adapted to fluidically connect a media supply filter test device to the coupling device. In addition, the at least one second connector is adapted to fluidically connect a multiplier device to the coupling device. Furthermore, the at least one third connector is configured as a hot steam connector. The at least one first connector, the at least one second connector and the at least one third connector are flow connected to the valve device, wherein the valve device is adapted to simultaneously flow connect at least one third connector of the at least one third connector to at least one first connector of the at least one first connector and to at least one second connector of the at least one second connector in a first operating mode, so that the media supply filter test device connected to the coupling device and the multiplier device connected to the coupling device can be sanitized simultaneously by means of the hot steam supplied through the at least one third connector.

Advantageously, it is possible by means of the coupling device to sanitize the media supply filter test device and the multiplier device simultaneously and, in particular, without fluidically decoupling the media supply filter test device from the coupling device.

In addition, advantageously, the coupling device, in particular media lines of the coupling device, can also be at least partially, preferably completely, sanitized in the first operating mode.

In a preferred embodiment, the coupling device comprises a plurality of first connectors, particularly preferably five or six first connectors. Alternatively or additionally, the coupling device preferably comprises a plurality of second connectors, particularly preferred three or four second connectors. Alternatively or additionally, the coupling device preferably comprises a third connector, in particular a hot steam connector.

In a further preferred embodiment, the coupling device can be operated in a plurality of first operating modes. In particular, the various first operating modes differ in the number of first and second connectors to which the at least one third connector is flow connected. In particular, the at least one third connector can be flow-connected to exactly one first connector and to exactly one second connector, or the at least one third connector can be flow-connected to a plurality of first connectors and/or a plurality of second connectors. In particular, the at least one third connector is flow-connected separately from all connectors and possibly an outlet to which it is not expressly connected in the manner described. Alternatively or additionally, the coupling device, in particular the valve device, is adapted to switch between at least two of the first operating modes.

In a further preferred embodiment, the media supply filter test device is an AquaWIT device of the company PALL, in particular in an embodiment available on the day determining the priority date of the present property right. Alternatively or additionally, the multiplier device is preferably a MUX device of the company PALL, in particular in an embodiment available on the day determining the priority date of the present property right. In particular, it is advantageously possible by means of the coupling device to simultaneously sanitize the AquaWIT device, the MUX device and, in particular, the fluidic connections between the AquaWIT device and the MUX device without first fluidically separating the AquaWIT device and the MUX device from the coupling device.

According to a further development of the invention, it is provided that the valve device is adapted to fluidically connect at least one first connector of the at least one first connector and at least one second connector of the at least one second connector to each other in a second operating mode and to fluidically separate each of them from the at least one third connector, so that a medium, in particular water and/or compressed air, can be routed from the media supply filter test device to the multiplier device.

Advantageously, it is thus possible to operate the multiplier device with the medium from the media supply filter test device without separating the coupling device from a sanitizing device providing, in particular, the hot steam.

In a preferred embodiment, the coupling device can be operated in a plurality of second operating modes. In particular, the various second operating modes differ in the number of first and second connectors that are flow connected to each other. In particular, exactly one first connector can be flow connected to exactly one second connector. Alternatively, a plurality of first connectors can be flow connected to exactly one second connector. Alternatively, a plurality of second connectors can be flow connected to exactly one first connector. Alternatively, a plurality of first connectors can be flow connected to a plurality of second connectors. In particular, the at least one first connector and the at least one second connector are flow-connected separately from all connectors and possibly an outlet to which they are not expressly connected in the manner described. Alternatively or additionally, the coupling device, in particular the valve device, is adapted to switch between at least two of the second operating modes.

In a further preferred embodiment, the coupling device comprises exactly one and only one third connector.

According to a further development of the invention, it is provided that the coupling device comprises at least one outlet. The at least one outlet is flow connected to the valve device. In an outlet mode, the valve device is adapted to fluidically connect the at least one outlet to at least one connector selected from at least one first connector of the at least one first connector, at least one second connector of the at least one second connector and at least one third connector of the at least one third connector, so that a medium present in the coupling device can escape from the coupling device via the at least one outlet.

Advantageously, it is thus possible in a simple manner to discharge a medium present in the coupling device and in particular in the multiplier device and/or the media supply filter test device, in particular excess medium.

In a preferred embodiment, the coupling device comprises exactly one and only one outlet.

In a further preferred embodiment, the coupling device can be operated in a plurality of outlet modes. In particular, the various outlet modes differ in the number of first, second and third connectors to which the at least one outlet is respectively flow connected. In particular, the at least one outlet can be flow connected to exactly one first connector or to a plurality of first connectors. Alternatively or additionally, the at least one outlet can be flow connected to exactly one second connector or to a plurality of second connectors. Alternatively or additionally, the at least one outlet can be flow connected to exactly one third connector or to a plurality of third connectors. In particular, the at least one outlet is flow-connected separately from all connectors to which it is not expressly connected in the manner described. Alternatively or additionally, the coupling device, in particular the valve device, is adapted to switch between at least two of the outlet modes.

In a further preferred embodiment, in the first operating mode, in particular in at least one first operating mode of the plurality of first operating modes, in particular in all first operating modes, the at least one outlet may be flow connected to the at least one third connector. In particular, the valve device is thus adapted to fluidically separate the at least one outlet from the at least one third connector in the first operating mode, in particular in at least one first operating mode of the plurality of first operating modes, in particular in all first operating modes, and/or to fluidically connect it to the at least one third connector. Advantageously, this makes it possible to sanitize at least one media line connecting the valve device to the at least one outlet in a first operating mode.

According to a further development of the invention, it is provided that the coupling device comprises at least one fourth connector. The at least one fourth connector is configured as a compressed air connector, wherein the at least one fourth connector is flow connected to the valve device. The valve device is adapted to connect, in a third operating mode, the at least one fourth connector to at least one first connector of the at least one first connector and/or to at least one second connector of the at least one second connector, so that the media supply filter test device connected to the coupling device and/or the multiplier device connected to the coupling device can be dried simultaneously by means of the compressed air supplied through the at least one fourth connector.

Advantageously, it is thus possible to dry the media supply filter test device and/or the multiplier device, in particular after sanitization and/or a filter test—in particular in simultaneously—without decoupling the coupling device from the media supply filter test device and the multiplier device in terms of flow.

In a preferred embodiment, the coupling device comprises exactly one and only one fourth connector.

In a further preferred embodiment, the coupling device is adapted to split the compressed air introduced by means of the at least one fourth connector such that a first portion of the compressed air is routed into the valve device and that a second portion of the compressed air is used as control air to actuate the valve device.

In a further preferred embodiment, the coupling device can be operated in a plurality of third operating modes. In particular, the various third operating modes differ in the number of first, second and third connectors to which the at least one fourth connector is respectively flow connected. In particular, the at least one fourth connector can be flow connected to exactly one first connector or to a plurality of first connectors. Alternatively or additionally, the at least one fourth connector can be flow connected to exactly one second connector or to a plurality of second connectors. Alternatively or additionally, the at least one fourth connector can be flow connected to exactly one third connector or to a plurality of third connectors. Alternatively or additionally, the at least one fourth connector can be flow connected to exactly one outlet or to a plurality of outlets. In particular, the at least one fourth connector is flow-connected separately from all connectors to which it is not expressly connected in the manner described. Alternatively or additionally, the coupling device, in particular the valve device, is adapted to switch between at least two of the third operating modes.

In a further preferred embodiment, in the first operating mode, in particular in at least one first operating mode of the plurality of first operating modes, in particular in all first operating modes, the at least one fourth connector may be flow connected to the at least one third connector. In particular, the valve device is thus adapted to fluidically separate the at least one fourth connector from the at least one third connector in the first operating mode, in particular in at least one first operating mode of the plurality of first operating modes, in particular in all first operating modes, and/or to fluidically connect it to the at least one third connector. Advantageously, this makes it possible to sanitize at least one media line, which connects the valve device to the at least one fourth connector, in a first operating mode.

In a further preferred embodiment, in the outlet mode, in particular in at least one outlet mode of the plurality of outlet modes, in particular in all outlet modes, the at least one fourth connector can be flow connected to the at least one outlet. In particular, the valve device is thus adapted to fluidically separate the at least one fourth connector from the at least one outlet in the outlet mode, in particular in at least one outlet mode of the plurality of outlet modes, in particular in all outlet modes, and/or to fluidically connect it to the at least one outlet.

According to a further development of the invention, it is provided that the coupling device comprises at least one fifth connector. The at least one fifth connector is configured as a water connector, wherein the at least one fifth connector is flow connected to the valve device. In a fourth operating mode, the valve device is adapted to flow-connect the at least one fifth connector to at least one first connector of the at least one first connector, so that the media supply filter test device connected to the coupling device can be filled by means of the water supplied through the at least one fifth connector.

Advantageously, the media supply filter test device can be filled with water by means of the at least one fifth connector without the media supply filter test device having to be fluidically separated from the coupling device beforehand.

In a preferred embodiment, the coupling device comprises exactly one and only one fifth connector.

In a further preferred embodiment, the coupling device can be operated in a plurality of fourth operating modes. In particular, the various fourth operating modes differ in the number of first connectors to which the at least one fifth connector is flow connected. In particular, the at least one fifth connector can be flow connected to exactly one first connector or to a plurality of first connectors. In particular, the at least one fifth connector is flow-connected separately from all connectors to which it is not expressly connected in the manner described. Alternatively or additionally, the coupling device, in particular the valve device, is adapted to switch between at least two of the fourth operating modes.

In a further preferred embodiment, in the first operating mode, in particular in at least one first operating mode of the plurality of first operating modes, in particular in all first operating modes, the at least one fifth connector may be flow connected to the at least one third connector. In particular, the valve device is thus adapted to fluidically separate the at least one fifth connector from the at least one third connector in the first operating mode, in particular in at least one first operating mode of the plurality of first operating modes, in particular in all first operating modes, and/or to fluidically connect it to the at least one third connector. Advantageously, this makes it possible to sanitize at least one media line, which connects the valve device to the at least one fifth connector, in a first operating mode.

In a further preferred embodiment, in the outlet mode, in particular in at least one outlet mode of the plurality of outlet modes, in particular in all outlet modes, the at least one fifth connection element can be flow connected to the at least one outlet. In particular, the valve device is thus adapted to fluidically separate the at least one fifth connector from the at least one outlet in the outlet mode, in particular in at least one outlet mode of the plurality of outlet modes, in particular in all outlet modes, and/or to fluidically connect it to the at least one outlet.

In a further preferred embodiment, in the third operating mode, in particular in at least one third operating mode of the plurality of third operating modes, in particular in all third operating modes, the at least one fifth connector can be flow connected to the at least one fourth connector. In particular, the valve device is thus adapted to fluidically separate the at least one fifth connector from the at least one fourth connector in the third operating mode, in particular in at least one third operating mode of the plurality of third operating modes, in particular in all third operating modes, and/or to fluidically connect it to the at least one fourth connector. It is thus advantageously possible to dry at least one media line, which connects the valve device to the at least one fifth connector, in a third operating mode by means of compressed air.

According to a further development of the invention, it is provided that the coupling device comprises at least one sixth connector. The at least one sixth connector is configured as a liquid supply connector, in particular as a lye supply connector, wherein the at least one sixth connector is flow connected to the valve device. In a fifth operating mode, the valve device is adapted to flow-connect at least one sixth connector of the at least one sixth connector to at least one first connector of the at least one first connector and/or to at least one second connector of the at least one second connector, so that the media supply filter test device connected to the coupling device and/or the multiplier device connected to the coupling device can be treated simultaneously by means of the liquid, in particular lye, supplied through the at least one sixth connector.

Advantageously, it is possible to treat the media supply filter test device and/or the multiplier device in a germ-killing and/or cleaning manner by means of a supplied lye solution. In addition, advantageously, in the fifth operating mode, the coupling device, in particular the media lines of the coupling device, can also be at least partially treated in a germicidal and/or cleaning manner.

In a preferred embodiment, the coupling device comprises exactly one and only one sixth connector.

In a further preferred embodiment, the coupling device is configured to be stable against lye, in particular concentrated sodium hydroxide solution.

In a further preferred embodiment, the coupling device can be operated in a plurality of fifth operating modes. In particular, the various fifth operating modes differ in the number of first and second connectors to which the at least one sixth connector is flow connected. In particular, the at least one sixth connector can be flow connected to exactly one first connector or to a plurality of first connectors. Alternatively or additionally, the at least one sixth connector can be flow connected to exactly one second connector or to a plurality of second connectors. In addition, the at least one sixth connector may, in particular, be flow connected to at least one connector selected from the at least one outlet, the at least one fourth connector and the at least one fifth connector. In particular, the at least one fifth connector is flow-connected separately from all connectors to which it is not expressly connected in the manner described. Alternatively or additionally, the coupling device, in particular the valve device, is adapted to switch between at least two of the fifth operating modes.

In a further preferred embodiment, in the first operating mode, in particular in at least one first operating mode of the plurality of first operating modes, in particular in all first operating modes, the at least one sixth connector may be flow connected to the at least one third connector. In particular, the valve device is thus adapted to fluidically separate the at least one sixth connector from the at least one third connector in the first operating mode, in particular in at least one first operating mode of the plurality of first operating modes, in particular in all first operating modes, and/or to fluidically connect it to the at least one third connector. Advantageously, this makes it possible to sanitize at least one media line connecting the valve device to the at least one sixth connector in a first operating mode.

In a further preferred embodiment, in the outlet mode, in particular in at least one outlet mode of the plurality of outlet modes, in particular in all outlet modes, the at least one sixth connector can be flow connected to the at least one outlet. In particular, the valve device is thus adapted to fluidically separate the at least one sixth connector from the at least one outlet in the outlet mode, in particular in at least one outlet mode of the plurality of outlet modes, in particular in all outlet modes, and/or to fluidically connect it to the at least one outlet.

In a further preferred embodiment, in the third operating mode, in particular in at least one third operating mode of the plurality of third operating modes, in particular in all third operating modes, the at least one sixth connector can be flow connected to the at least one fourth connector. In particular, the valve device is thus adapted to fluidically separate the at least one sixth connector from the at least one fourth connector in the third operating mode, in particular in at least one third operating mode of the plurality of third operating modes, in particular in all third operating modes, and/or to fluidically connect it to the at least one fourth connector. It is thus advantageously possible to dry at least one media line, which connects the valve device to the at least one sixth connector, in a third operating mode by means of compressed air.

According to a further development of the invention, it is provided that the valve device comprises at least one valve, preferably a plurality of valves. The at least one valve is arranged fluidically between the at least one first connector, the at least one second connector and the at least one third connector and is adapted to control the flow within the coupling device.

In a preferred embodiment, an operating mode of the valve device can be set by controlling the at least one valve.

According to a further development of the invention, it is provided that the at least one valve comprises at least one membrane comprising Polytetrafluorethylene (PTFE).

According to a further development of the invention, it is provided that the coupling device comprises a control device. The control device is adapted to set an operating mode of the valve device.

In a preferred embodiment, the control device is operatively connected to the valve device and adapted to actuate the valve device, in particular the at least one valve, in order to set an operating mode.

According to a further development of the invention, it is provided that the coupling device comprises at least one seventh connector. The at least one seventh connector is configured as a pneumatic connector, wherein the at least one seventh connector is flow connected to the valve device. The valve device is adapted to be controlled via the at least one seventh connector in order to set an operating mode.

In a preferred embodiment, the at least one valve is adapted to be actuated via the at least one seventh connector to set an operating mode.

In a further preferred embodiment, the coupling device comprises a plurality of seventh connectors, particularly preferably twelve or thirteen seventh connectors.

In particular, each seventh connector is connected to at least one valve, so that the valve can be controlled by means of the respective seventh connector.

According to a further development of the invention, it is provided that the coupling device comprises at least one pressure reducing device. The pressure reducing device is flow connected to the at least one fourth connector.

In a preferred embodiment, the pressure reducing device preferably comprises a pressure gauge adapted to indicate a pressure of the compressed air supplied via the fourth connector.

According to a further development of the invention, it is provided that the coupling device comprises a first connecting element. The first connecting element is adapted to mechanically—in particular rigidly—connect the media supply filter test device to the coupling device.

In a preferred embodiment, the first connecting element is configured such that the media supply filter test device connected to the coupling device by means of the first connecting element is not movable relative to the coupling device.

According to a further development of the invention, it is provided that the coupling device comprises a second connecting element. The second connecting element is adapted to mechanically—in particular rigidly—connect the multiplier device to the coupling device.

In a preferred embodiment, the second connecting element is configured such that the multiplier device connected to the coupling device by means of the second connecting element is not movable relative to the coupling device.

According to a further development of the invention, it is provided that at least one connecting element, selected from the first connecting element and the second connecting element, comprises a positioning element. The positioning element is preferably adapted to align and position the media supply filter test device and/or the multiplier device relative to the coupling device in a simple manner.

According to a further development of the invention, it is provided that the coupling device comprises a fastening device. The fastening device is adapted to fasten the coupling device in a base and/or on a wall.

According to a further development of the invention, it is provided that the coupling device comprises at least one cover element. The at least one cover element is adapted to cover at least one line element, in particular a hose, which fluidically connects the coupling device to at least one device, in particular selected from the media supply filter test device and the multiplier device.

In a particularly preferred embodiment, the coupling device comprises a first cover element and a second cover element. The first cover element comprises the first connecting element with a first positioning element. The second cover element comprises the second connecting element with a second positioning element.

According to a further development of the invention, it is provided that the coupling device comprises at least one eighth connector, wherein the at least one eighth connector is adapted to fluidically connect at least one device using compressed air, in particular a filter drying device, to the coupling device. The at least one eighth connector is flow connected to at least one fourth connector of the at least one fourth connector in order to apply compressed air to the at least one device using compressed air.

In a preferred embodiment, the coupling device comprises two eighth connectors.

In a further preferred embodiment, the at least one eighth connector is flow connected to at least one fourth connector of the at least one fourth connector via a first media line. In addition, the media line is preferably flow-connected to a second media line that flow-connects the at least one fourth connector to the pressure reducing device, so that at least a portion of the compressed air introduced through the at least one fourth connector is routed to the at least one eighth connector before the compressed air enters the pressure reducing device.

In a further preferred embodiment, the at least one eighth connector is flow connected to at least one fourth connector of the at least one fourth connector via the valve device, and the valve device is adapted to apply compressed air to the at least one device using compressed air in a sixth operating mode.

In a further preferred embodiment, the coupling device can be operated in a plurality of sixth operating modes. The various sixth operating modes differ in particular in the number of fourth and eighth connectors that are flow connected to each other. In particular, exactly one fourth connector can be flow connected to exactly one eighth connector. Alternatively, a plurality of fourth connectors can be flow connected to exactly one eighth connector. Alternatively, a plurality of eighth connectors can be flow connected to exactly one fourth connector. Alternatively, a plurality of fourth connectors can be flow connected to a plurality of eighth connectors. In particular, the at least one fourth connector and the at least one eighth connector are flow-connected separately from all connectors and possibly an outlet to which they are not expressly connected in the manner described. Alternatively or additionally, the coupling device, in particular the valve device, is adapted to switch between at least two of the six operating modes.

In a further preferred embodiment, in the first operating mode, in particular in at least one first operating mode of the plurality of first operating modes, in particular in all first operating modes, the at least one eighth connector is fluidically separated from the at least one third connector. This advantageously makes it possible to prevent sanitization of the at least one eighth connector. Alternatively, the at least one eighth connector can be flow connected to the at least one third connector by means of the flow connection via the valve device in the first operating mode, in particular in at least one first operating mode of the plurality of first operating modes, in particular in all first operating modes. Advantageously, this makes it possible to sanitize at least one media line connecting the valve device to the at least one eighth connector in a first operating mode. In particular, the valve device is adapted to fluidically separate the at least one eighth connector from the at least one third connector in the first operating mode, in particular in at least one first operating mode of the plurality of first operating modes, in particular in all first operating modes, or to fluidically connect it to the at least one third connector.

In a further preferred embodiment, in the outlet mode, in particular in at least one outlet mode of the plurality of outlet modes, in particular in all outlet modes, the at least one eighth connector can be flow connected to the at least one outlet. In particular, the valve device is thus adapted to fluidically separate the at least one eighth connector from the at least one outlet in the outlet mode, in particular in at least one outlet mode of the plurality of outlet modes, in particular in all outlet modes, and/or to fluidically connect it to the at least one outlet.

In a further preferred embodiment, in the third operating mode, in particular in at least one third operating mode of the plurality of third operating modes, in particular in all third operating modes, the at least one eighth connector can be flow connected to the at least one fourth connector. In particular, the valve device is thus adapted to fluidically separate the at least one eighth connector from the at least one fourth connector in the third operating mode, in particular in at least one third operating mode of the plurality of third operating modes, in particular in all third operating modes, and/or to fluidically connect it to the at least one fourth connector. This advantageously makes it possible to dry at least one media line, which connects the valve device to the at least one eighth connector, in a third operating mode by means of compressed air.

In a further preferred embodiment, in the fifth operating mode, in particular in at least one fifth operating mode of the plurality of fifth operating modes, in particular in all fifth operating modes, the at least one eighth connector is fluidically separated from the at least one sixth connector. This advantageously makes it possible to prevent at least partial germicidal and/or cleaning treatment of the at least one eighth connector. Alternatively, the at least one eighth connector can be flow connected to the at least one sixth connector in the fifth operating mode, in particular in at least one fifth operating mode of the plurality of fifth operating modes, in particular in all fifth operating modes, by means of the flow connection via the valve device. It is thus advantageously possible to at least partially treat at least one media line, which connects the valve device to the at least one eighth connector, in a fifth operating mode, in particular by means of a supplied lye in a germ-killing and/or cleaning manner. In particular, the valve device is thus adapted to fluidically separate the at least one eighth connector from the at least one sixth connector in the fifth operating mode, in particular in at least one fifth operating mode of the plurality of fifth operating modes, in particular in all fifth operating modes, or to fluidically connect it to the at least one sixth connector.

In one embodiment, an operating mode selected from the first operating mode, the second operating mode, the third operating mode, the fourth operating mode, the fifth operating mode, the sixth operating mode and the outlet mode is selected by means of the control device. At least one first valve of the valve device is actuated by means of the control device in order to set the selected operating mode. In addition, preferably at least one second valve of the valve device, different in particular from the at least one first valve, is actuated by means of the at least one seventh connector, wherein a partial operating mode of the selected operating mode can be set thereby.

In a further particularly preferred embodiment, at least one connector, selected from the at least one first connector, the at least one second connector, the at least one third connector, the at least one fourth connector, the at least one fifth connector, the at least one sixth connector, the at least one seventh connector and the at least one eighth connector, is configured as a plug-in coupling, in particular as a Stäubli connector, in particular for Teflon hoses.

The task is also solved by creating a filter test device with a coupling device according to the invention or a coupling device according to one or more of the embodiments explained above. In addition, the filter test device comprises a media supply filter test device and a multiplier device. The media supply filter test device is flow connected to the coupling device via the at least one first connector. Furthermore, the multiplier device is flow connected to the coupling device via the at least one second connector. In particular, the advantages already explained in connection with the coupling device arise in connection with the filter test device.

In a particularly preferred embodiment, the media supply filter test device is an AquaWIT device from the company PALL, in particular in an embodiment available on the day determining the priority date of the present property right. Alternatively or additionally, the multiplier device is preferably a MUX device of the company PALL, in particular in an embodiment available on the day determining the priority date of the present IP right.

In particular, the at least one seventh connector configured as a pneumatic connector is flow connected to the media supply filter test device, in particular the AquaWIT device, so that the valve device of the coupling device can be controlled by means of the media supply filter test device. Alternatively or additionally, in particular the at least one seventh connector configured as a pneumatic connector is flow connected to the multiplier device, in particular the MUX device, so that the valve device of the coupling device can be controlled by means of the multiplier device.

The media supply filter test device is mechanically connected to the coupling device, in particular by means of the first connecting element, in particular in such a way that the media supply filter test device and the coupling device are not movable relative to each other.

The multiplier device is mechanically connected to the coupling device, in particular by means of the second connecting element, in particular in such a way that the multiplier device and the coupling device are not movable relative to each other.

In one embodiment, the filter test device also comprises at least one device using compressed air, in particular a filter drying device, which is flow connected to the coupling device via the at least one eighth connector. In particular, the at least one eighth connector is flow connected to the at least one fourth connector of the at least one fourth connector in order to apply compressed air to the at least one device using compressed air.

Further areas of applicability will become apparent from the description provided herein. The description and specific examples in this summary are intended for purposes of illustration only and are not intended to limit the scope of the present disclosure.

### DRAWINGS

The drawings described herein is for illustrative purposes only of selected embodiments and not all possible implementations, and are not intended to limit the scope of the present disclosure.

The invention is explained in more detail with reference to the following drawing. Thereby shows:

FIG. 1 a schematic representation of a first embodiment example of a coupling device,

FIG. 2 a schematic representation of a second embodiment example of the coupling device,

FIG. 3 a schematic representation of a third embodiment example of the coupling device,

FIG. 4 a schematic representation of a fourth embodiment example of the coupling device,

FIG. 5 a schematic representation of a fifth embodiment example of the coupling device,

FIG. 6 a schematic representation of a sixth embodiment example of the coupling device,

FIG. 7 a schematic representation of a seventh embodiment example of the coupling device,

FIG. 8 a schematic representation of an eighth embodiment example of the coupling device,

FIG. 9 a schematic representation of a ninth embodiment example of the coupling device,

FIG. 10 a schematic representation of a tenth embodiment example of the coupling device,

FIG. 11 a schematic representation of a first embodiment example of a filter test device,

FIG. 12 a schematic representation of a second embodiment example of the filter test device,

FIG. 13 a schematic representation of a third embodiment example of the filter test device, and

FIG. 14 a schematic representation of a fourth embodiment example of the filter test device.

### DETAILED DESCRIPTION

Example embodiments will now be described more fully with reference to the accompanying drawings.

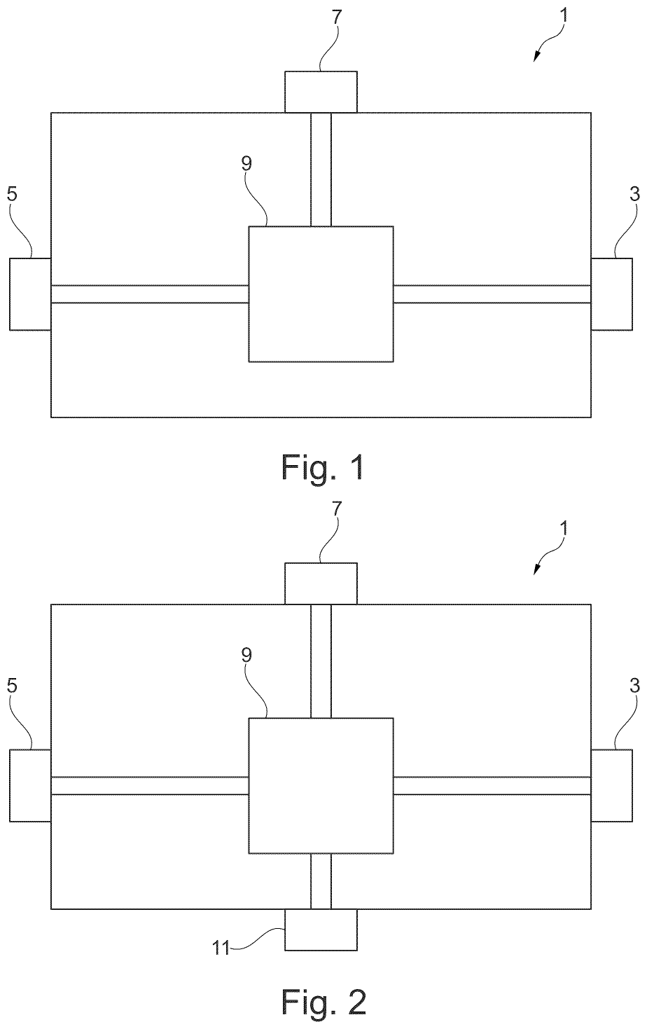

FIG. 1 shows a schematic representation of a first embodiment example of a coupling device 1. The coupling device comprises a first connector 3, a second connector 5, a third connector 7 and a valve device 9. The first connector 3, the second connector 5 and the third connector 7 are flow connected to the valve device 9.

The first connector 3 is adapted to fluidically connect a media supply filter test device to the coupling device 1. The second connector 5 is adapted to fluidically connect a multiplier device to the coupling device 1. The third connector 7 is configured as a hot steam connector.

The valve device 9 is adapted to connect the third connector 7 to the first connector 3 and to the second connector 5 simultaneously in a first operating mode, so that the media supply filter test device connected to the coupling device 1 and the multiplier device connected to the coupling device 1 can be sanitized simultaneously by means of the hot steam supplied through the third connector 7.

In particular, the valve device 9 is additionally adapted to fluidically connect and/or disconnect the first connector 3, the second connector 5 and the third connector 7.

FIG. 2 shows a schematic representation of a second embodiment example of the coupling device 1.

Identical and functionally identical elements are provided with identical reference signs in all figures, so that reference is made to the previous description.

The coupling device 1 also preferably comprises an outlet 11, wherein the outlet 11 is flow connected to the valve device 9.

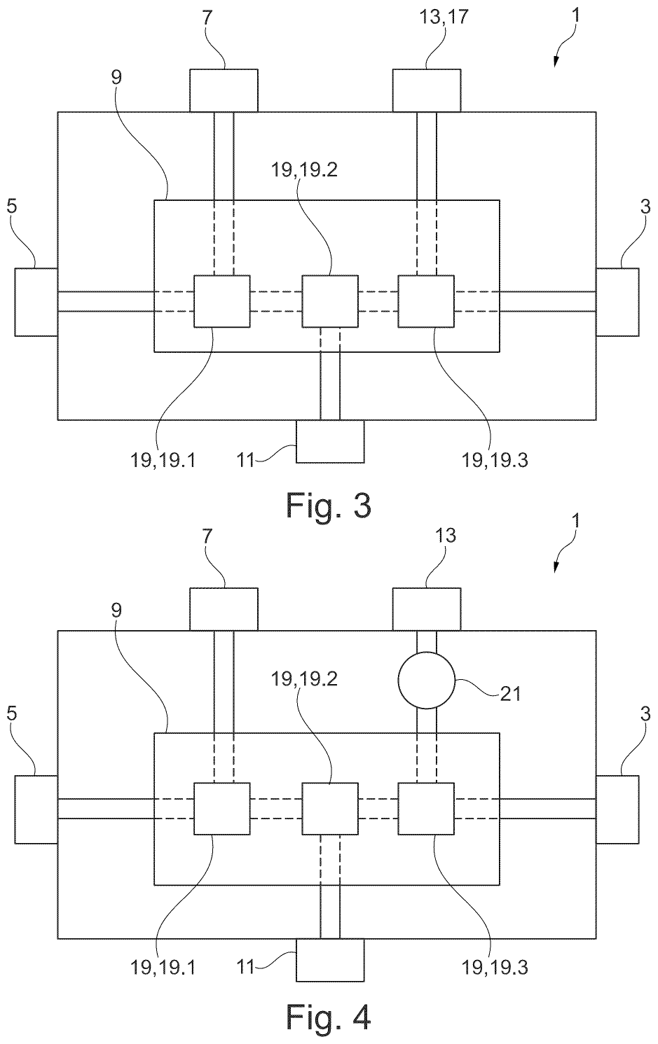

FIG. 3 shows a schematic representation of a third embodiment example of the coupling device 1.

The coupling device 1 additionally preferably comprises a further connector selected from a fourth connector 13, in particular a compressed air connector, and a sixth connector 17, in particular a liquid supply connector.

The valve device 9 additionally preferably comprises at least one valve 19, in particular a first valve 19.1, a second valve 19.2 and a third valve 19.3. The valve 19, in particular the first valve 19.1, the second valve 19.2 and the third valve 19.3, is adapted to fluidically connect and/or disconnect the first connector 3, the second connector 5, the third connector 7, the outlet 11 and the further connector selected from the fourth connector 13 and the sixth connector 17.

FIG. 4 shows a schematic representation of a fourth embodiment example of the coupling device 1.

In addition to the connectors shown in FIG. 2, the coupling device 1 comprises the fourth connector 13 and a pressure reducing device 21. The pressure reducing device 21 is flow connected to the fourth connector 13.

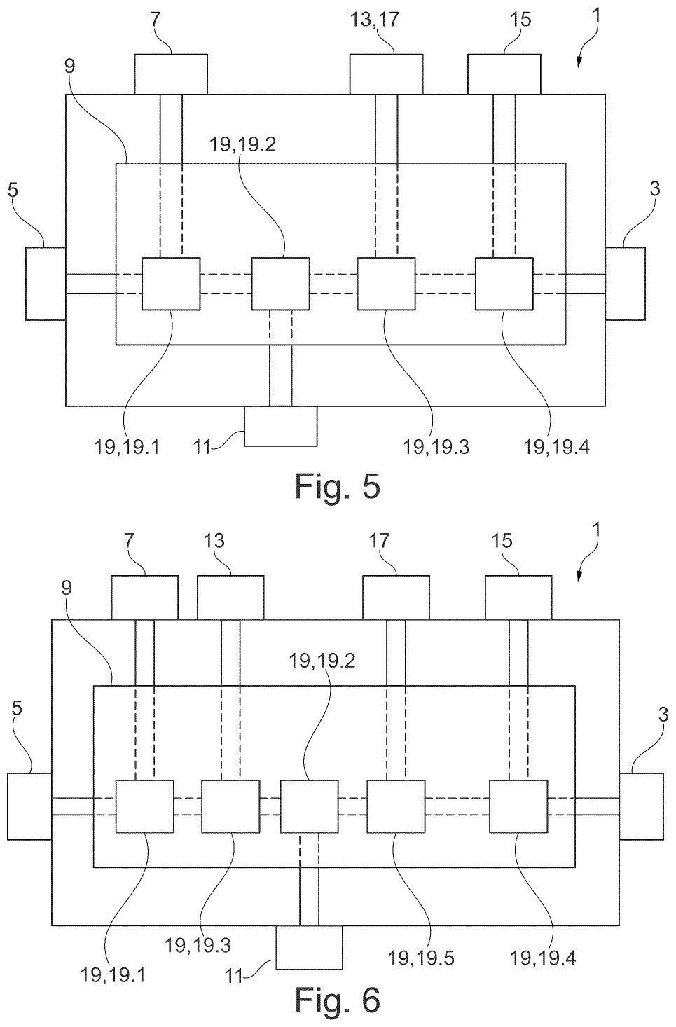

FIG. 5 shows a schematic representation of a fifth embodiment example of the coupling device 1.

In addition to the connectors shown in FIG. 3, the coupling device 1 comprises a fifth connector 15, in particular a water connector, and a fourth valve 19.4.

FIG. 6 shows a schematic representation of a sixth embodiment example of the coupling device 1.

In addition to the first connector 3, the second connector 5 and the third connector 7, the coupling device 1 comprises the outlet 11, the fourth connector 13, the fifth connector 15 and the sixth connector 17. Furthermore, the valve device 9 comprises five valves, in particular the first valve 19.1, the second valve 19.2, the third valve 19.3, the fourth valve 19.4 and a fifth valve 19.5.

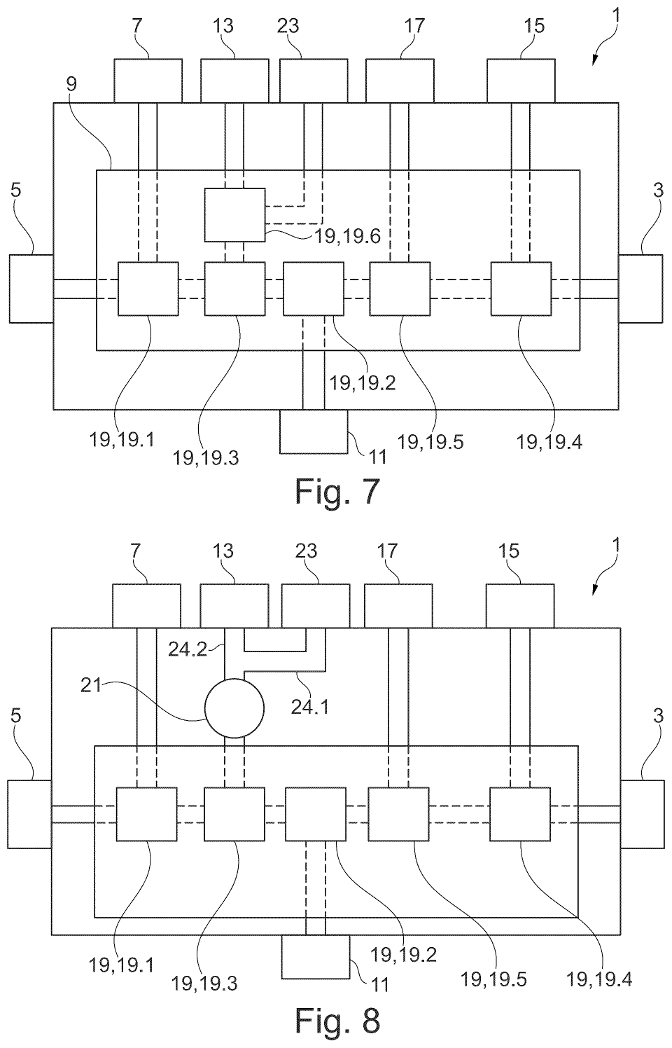

FIG. 7 shows a schematic representation of a seventh embodiment example of the coupling device 1.

In addition to the coupling device of FIG. 6, the coupling device 1 comprises an eighth connector 23 and a sixth valve 19.6, wherein compressed air from the fourth connector 13 can be supplied to a filter drying device in particular by means of the eighth connector 23.

FIG. 8 shows a schematic representation of an eighth embodiment example of the coupling device 1.

In addition to the coupling device of FIG. 6, the coupling device 1 comprises the eighth connector 23 and the pressure reducing device 21, wherein compressed air from the fourth connector 13 can be supplied to a filter drying device in particular by means of the eighth connector 23.

In particular, the eighth connector 23 is flow connected to the fourth connector 13 via a first media line 24.1. In addition, the first media line 24.1 is flow connected to a second media line 24.2, which flow connects the fourth connector 13 to the pressure reducing device 21, so that at least part of the compressed air introduced through the fourth connector 13 is routed to the eighth connector 23 before the compressed air enters the pressure reducing device 21.

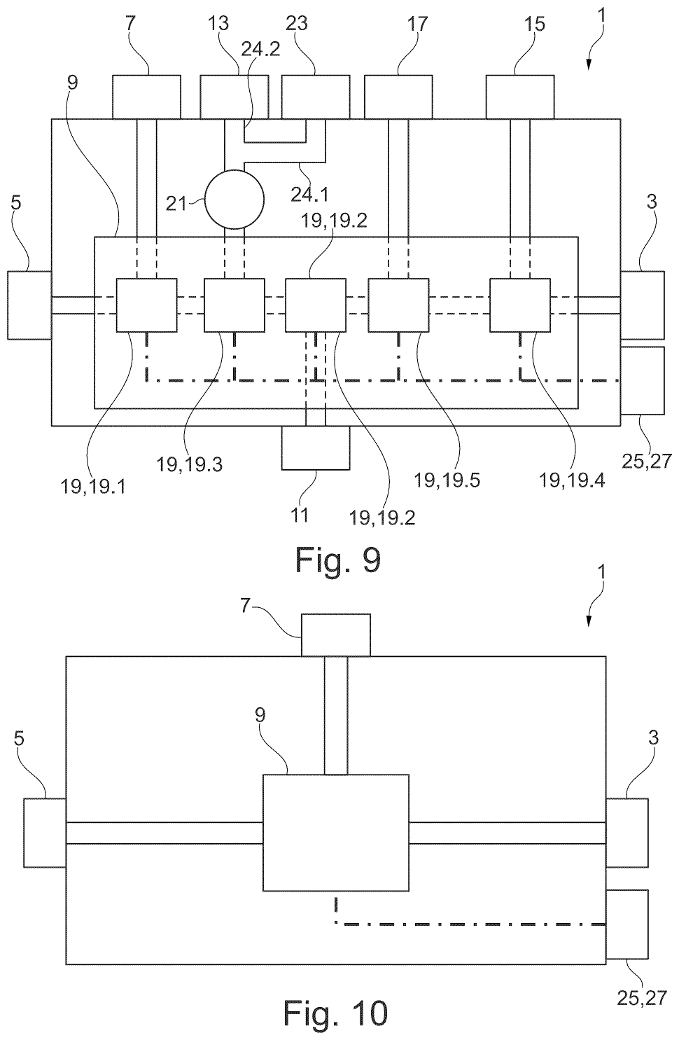

FIG. 9 shows a schematic representation of an eighth embodiment example of the coupling device 1.

In addition to the coupling device of FIG. 8, the coupling device 1 comprises a device selected from a seventh connector 25, in particular a pneumatic connector, and a control device 27.

The device, selected from the seventh connector 25, in particular the pneumatic connector, and the control device 27, is operatively connected to the valve device 9, in particular the plurality of valves 19, and is adapted to control the valve device 9, in particular the plurality of valves 19.

FIG. 10 shows a schematic representation of a ninth embodiment example of the coupling device 1.

In addition to the coupling device of FIG. 1, the coupling device 1 comprises a device selected from the seventh connector 25, in particular the pneumatic connector, and the control device 27.

The valve devices 9 shown in FIGS. 1 to 10 are adapted to simultaneously connect the third connector to the first connector 3 and the second connector 5 in a first operating mode, so that a media supply filter test device connected to the coupling device 1 and a multiplier device connected to the coupling device 1 can be sanitized simultaneously by means of hot steam supplied through the third connector 7. Optionally, the valve device is additionally adapted to fluidically connect the third connector to at least one connector selected from the fourth connector 13, the fifth connector 15, the sixth connector 17, the eighth connector 23 and the outlet 11 in the first operating mode. In particular, all other connectors that are not flow connected to the third connector 7 are flow separated from the third connector 7.

Preferred, the valve devices 9 shown in FIGS. 1 to 10 are additionally adapted to fluidically connect the first connector 3 and the second connector 5 to each other in a second operating mode and to fluidically separate them from all other connectors, in particular from the third connector 7, the fourth connector 13, the fifth connector 15, the sixth connector 17, the eighth connector 23 and the outlet 11, so that a medium, in particular water and/or compressed air, can be routed from the media supply filter test device to the multiplier device by means of the coupling device 1.

Preferably, the valve devices 9 shown in FIGS. 3 to 9 are additionally adapted to simultaneously connect the fourth connector 13 to the first connector 3 and the second connector 5 in a third operating mode, so that the media supply filter test device connected to the coupling device 1 and the multiplier device connected to the coupling device 1 can be dried simultaneously by means of the compressed air supplied through the fourth connector 13. Preferably, the other connectors, in particular the third connector 7, the fifth connector 15, the sixth connector 17, the eighth connector 23 and the outlet 11, are each flow connected to the fourth connector 13 or separated from the fourth connector 13.

Preferred, the valve devices 9 shown in FIGS. 5 to 9 are additionally adapted to fluidically connect the fifth connector 15 to the first connector 3 in a fourth operating mode and preferably to fluidically separate it from the second connector 5, the third connector 7, the fourth connector 13, the sixth connector 17, the eighth connector 23 and the outlet 11, so that the media supply filter test device connected to the coupling device 1 can be filled by means of the water supplied through the fifth connector 13.

Preferred, the valve devices 9 shown in FIGS. 3 and 5 to 9 are additionally adapted to flow-connect the sixth connector 17 to the first connector 3 and the second connector 5 at the same time in a fifth operating mode, so that the media supply filter test device connected to the coupling device 1 and the multiplier device connected to the coupling device 1 can be treated simultaneously by means of the liquid, in particular lye, supplied through the sixth connector 17. In particular, all other connectors are in each case either flow connected to the sixth connector 17 or separated from the sixth connector 17.

Preferred, the valve device 9 shown in FIG. 7 is additionally adapted to flow-connect the eighth connector 23 to the fourth connector 13 in a sixth operating mode in order to conduct compressed air from the fourth connector 13 to a device using compressed air 35, in particular a filter drying device, which is connected to the coupling device 1 via the eighth connector 23. In particular, all other connectors are fluidically separated from the eighth connector 23.

Preferred, the valve devices 9 shown in FIGS. 2 to 9 are additionally adapted to fluidically connect the outlet 11 in an outlet mode to at least one connector selected from the first connector 3, the second connector 5, the third connector 7, the fourth connector 13, the fifth connector 15, the sixth connector 17 and the eighth connector 23, so that a medium present in the coupling device 1 can escape from the coupling device 1 via the outlet 11.

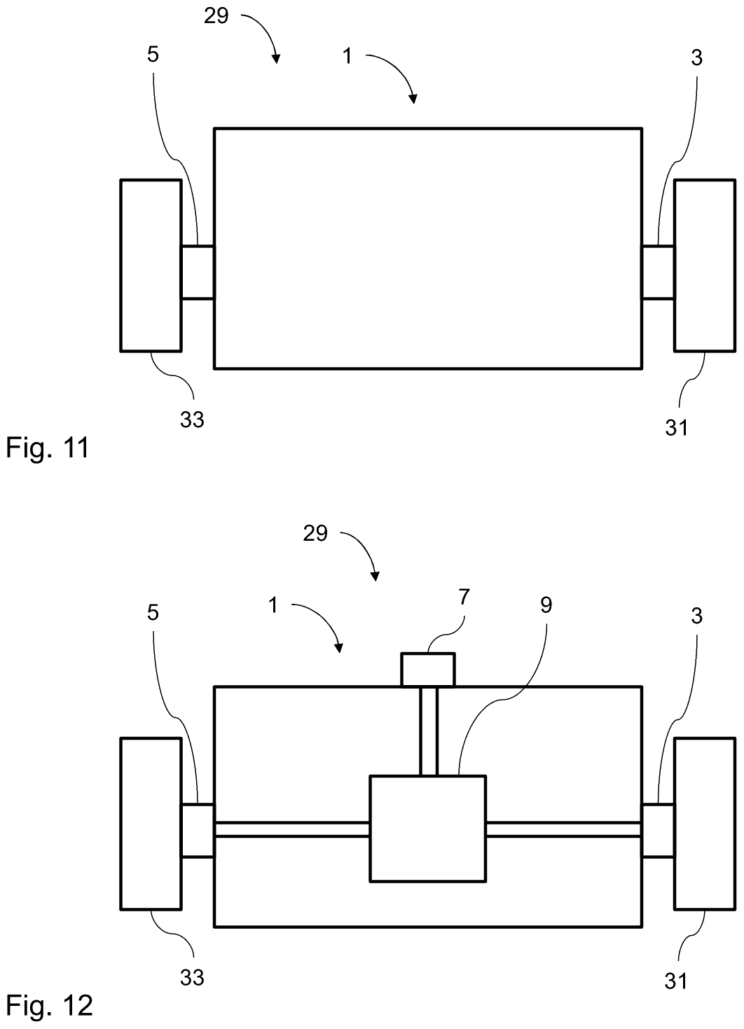

FIG. 11 shows a schematic representation of a first embodiment example of a filter test device 29.

The filter test device 29 comprises the coupling device 1, a media supply filter test device 31, in particular the AquaWIT device, and a multiplier device 33, in particular a MUX device. Here, the media supply filter test device 31, in particular the AquaWIT device, is flow connected to the coupling device 1 via the at least one first connector 3. In addition, the multiplier device 33, in particular a MUX device, is flow connected to the coupling device 1 via the at least one second connector 5.

In particular, the coupling device 1 is a coupling device 1 shown in at least one of FIGS. 1 to 10.

FIG. 12 shows a schematic representation of a second embodiment example of the filter test device 29.

The filter test device 29 comprises the coupling device 1 according to FIG. 1, the media supply filter test device 31, in particular the AquaWIT device, and the multiplier device 33, in particular a MUX device. Here, the media supply filter test device 31, in particular the AquaWIT device, is flow connected to the coupling device 1 via the at least one first connector 3. In addition, the multiplier device 33, in particular a MUX device, is flow connected to the coupling device 1 via the at least one second connector 5.

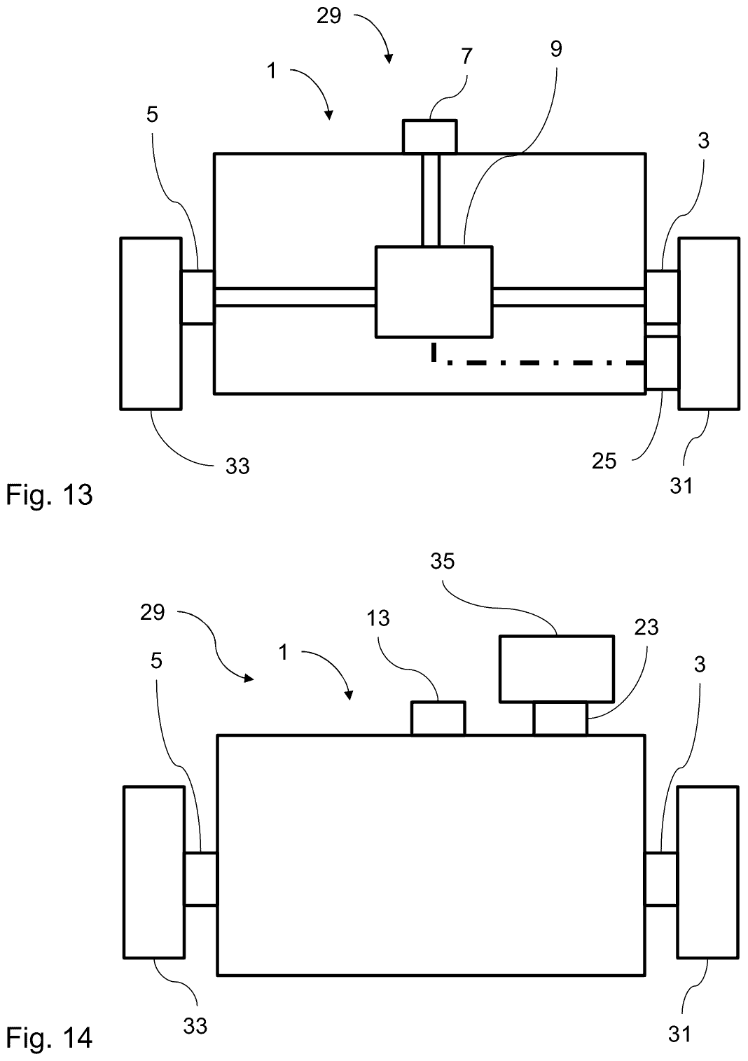

FIG. 13 shows a schematic representation of a third embodiment example of the filter test device 29.

The filter test device 29 comprises the coupling device 1 according to FIG. 10, the media supply filter test device 31, in particular the AquaWIT device, and the multiplier device 33, in particular a MUX device. Here, the media supply filter test device 31, in particular the AquaWIT device, is flow connected to the coupling device 1 via the at least one first connector 3. In addition, the multiplier device 33, in particular a MUX device, is flow connected to the coupling device 1 via the at least one second connector 5. Furthermore, the at least one seventh connector 25 configured as a pneumatic connector is flow connected to the media supply filter test device 31, in particular the AquaWIT device, so that the valve device 9 of the coupling device 1 is controlled by means of the media supply filter test device 31.

FIG. 14 shows a schematic representation of a fourth embodiment example of the filter test device 29.

The filter test device 29 comprises the coupling device 1, a media supply filter test device 31, in particular the AquaWIT device, and a multiplier device 33, in particular a MUX device, and a device using compressed air 35, in particular a filter drying device. Here, the media supply filter test device 31, in particular the AquaWIT device, is flow connected to the coupling device 1 via the at least one first connector 3. Furthermore, the multiplier device 33, in particular a MUX device, is flow connected to the coupling device 1 via the at least one second connector 5. In addition, the device using compressed air 35, in particular a filter drying device, is flow connected to the coupling device 1 via the at least one eighth connector 23. In particular, the at least one eighth connector 23 is flow connected to the at least one fourth connector 13 in order to apply compressed air to the at least one device using compressed air 35.

In particular, the coupling device 1 is a coupling device 1 shown in at least one of FIGS. 7 to 9.

The foregoing description of the embodiment has been provided for purposes of illustration and description. It is not intended to be exhaustive or to limit the disclosure. Individual elements or features of a particular embodiment are generally not limited to that particular embodiment, but, where applicable, are inter-changeable and can be used in a selected embodiment, even if not specifically shown or described. The same may also be varied in many ways. Such variations are to be regarded as a departure from the disclosure, and all such modifications are intended to be included within the scope of the disclosure.

1. A coupling device with
at least one first connector, at least one second connector, at least one third connector and a valve device, wherein
the at least one first connector is adapted to fluidically connect a media supply filter test device to the coupling device, wherein
the at least one second connector is adapted to fluidically connect a multiplier device to the coupling device, wherein
the at least one third connector is configured as a hot steam connector, wherein
the at least one first connector, the at least one second connector and the at least one third connector are flow connected to the valve device, wherein
the valve device is adapted to connect, in a first operating mode, at least one third connector of the at least one third connector fluidically simultaneously to at least one first connector of the at least one first connector and to at least one second connector of the at least one second connector, so that the media supply filter test device connected to the coupling device and the multiplier device connected to the coupling device can be sanitized simultaneously by means of the hot steam supplied through the at least one third connector.

2. The coupling device according to claim 1, wherein the valve device is adapted to fluidically connect at least one first connector of the at least one first connector and at least one second connector of the at least one second connector in a second operating mode and to fluidically separate each of them from the at least one third connector, so that a medium selected from a group consisting of, water, compressed air and a combination thereof is routable from the media supply filter test device to the multiplier device.

3. The coupling device according to claim 1 with at least one outlet, wherein
the at least one outlet is flow connected to the valve device, and wherein
the valve device is adapted to fluidically connect, in an outlet mode, at least one outlet of the at least one outlet to at least one connector selected from at least one first connector of the at least one first connector, and at least one second connector of the at least one second connector and at least one third connector of the at least one third connector, so that a medium present in the coupling device can escape from the coupling device via the at least one outlet.

4. The coupling device according to claim 1 with at least one fourth connector, wherein
the at least one fourth connector is configured as a compressed air connector, wherein
the at least one fourth connector is flow connected to the valve device, wherein
the valve device is adapted to connect, in a third operating mode, at least one fourth connector of the at least one fourth connector by flow to at least one first connector of the at least one first connector and/or to at least one second connector of the at least one second connector, so that the media supply filter test device connected to the coupling device and/or the multiplier device connected to the coupling device can be dried by means of the compressed air supplied through the at least one fourth connector.

5. The coupling device according to claim 1, comprising at least one fifth connector, wherein
the at least one fifth connector is configured as a water connector, wherein
the at least one fifth connector is flow connected to the valve device, wherein
the valve device is adapted to connect at least one fifth connector of the at least one fifth connector to at least one first connector of the at least one first connector in a fourth operating mode, so that the media supply filter test device connected to the coupling device can be filled by means of the water supplied through the at least one fifth connector.

6. The coupling device according to claim 1, comprising at least one sixth connector, wherein
the at least one sixth connector is configured as a liquid supply connector, in particular as a lye supply connector, wherein
the at least one sixth connector is flow connected to the valve device, wherein
the valve device is adapted to fluidically connect, in a fifth operating mode, at least one sixth connector of the at least one sixth connector to at least one first connector of the at least one first connector and/or to at least one second connector of the at least one second connector, so that the media supply filter test device connected to the coupling device and/or the multiplier device connected to the coupling device can be treated by means of the liquid, in particular lye, supplied through the at least one sixth connector.

7. The coupling device according to claim 1, wherein the valve device comprises at least one valve, preferably a plurality of valves, wherein the at least one valve is arranged fluidically between the at least one first connector, the at least one second connector and the at least one third connector and is adapted to control the flow within the coupling device.

8. The coupling device according to claim 1, wherein the at least one valve comprises at least one membrane comprising Polytetrafluorethylene (PTFE).

9. The coupling device according to claim 1, comprising a control device, wherein the control device is adapted to set an operating mode of the valve device, preferably the control device being operatively connected to the valve device and adapted to control the valve device, in particular the at least one valve, to set an operating mode.

10. The coupling device according to claim 1 with at least one seventh connector, wherein
the at least one seventh connector is configured as a pneumatic connector, wherein
the at least one seventh connector is flow connected to the valve device, wherein
the valve device, in particular the at least one valve, is adapted to be actuated via the at least one seventh connector in order to set an operating mode.

11. The coupling device according to claim 4, comprising at least one pressure reducing device, wherein
the pressure reducing device is flow connected to the at least one fourth connector, wherein
the pressure reducing device preferably comprises a pressure gauge adapted to indicate a pressure of the supplied compressed air.

12. The coupling device according to claim 1 with a first connecting element, wherein the first connecting element is adapted to mechanically connect the media supply filter test device to the coupling device, in particular to connect it in such a way that the media supply filter test device and the coupling device are not movable relative to each other.

13. The coupling device according to claim 1, comprising a second connecting element, wherein the second connecting element is adapted to mechanically connect the multiplier device to the coupling device, in particular to connect in such a way that the multiplier device and the coupling device are not movable relative to each other.

14. The coupling device according to claim 1, wherein at least one connecting element, selected from the first connecting element and the second connecting element, comprises a positioning element.

15. The coupling device according to claim 1, comprising a fastening device adapted to fasten the coupling device in a base and/or on a wall.

16. The coupling device according to claim 1 with at least one cover element, wherein the at least one cover element is adapted to cover at least one hose which fluidically connects the coupling device to at least one device, in particular selected from the media supply filter test device and the multiplier device.

17. The coupling device according to claim 1, comprising at least one eighth connector, wherein
the at least one eighth connector is adapted to fluidically connect at least one device using compressed air, in particular a filter drying device, to the coupling device, wherein
the at least one eighth connector is flow connected to at least one fourth connector of the at least one fourth connector in order to apply compressed air to the at least one device using compressed air.

18. A filter test device comprising a coupling device according to claim 1, a media supply filter test device, in particular the AquaWIT device, and a multiplier device, in particular a MUX device, wherein the media supply filter test device is flow connected to the coupling device via the at least one first connector, and wherein the multiplier device is flow connected to the coupling device via the at least one second connector.
